(19) United States
(12) Patent Application Publication
(10) Pub. No.: US 2011/0082364 A1
(43) Pub. Date: Apr. 7, 2011

(54) MRI Biopsy Targeting Cube with Retention Wiper

(76) Inventors: John A. Hibner, Mason, OH (US); Kreena Avimukta, Mason, OH (US); Kevin R. Shannon, Mason, OH (US)

(21) Appl. No.: 12/573,177

(22) Filed: Oct. 5, 2009

(51) Int. Cl. A61B 5/055; A61B 10/02

(52) U.S. Cl. 600/415; 600/562

(57) ABSTRACT

A biopsy system comprises a localization assembly, a biopsy device, and a targeting cube. At least a portion of the biopsy device is insertable through a targeting cube, which is insertable into a selected aperture of a grid plate. The targeting cube comprises a body defined by faces, with guide passageways that originate and terminate at the faces and pass through the body of the targeting cube to accommodate insertion of a part of the biopsy device. An exterior wiper retainer of the targeting cube engages the grid plate to permit insertion of the targeting cube into a selected aperture of the grid plate while resisting withdrawal of the targeting cube from the grid plate. Internal wiper retainers in the guide passageways permit insertion of part of the biopsy device through a selected guide passageway while resisting withdrawal of the inserted part from the targeting cube.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. Merely exemplary biopsy devices are disclosed in U.S. Pat. No. 6,273,862, entitled “Surgical Device for the Collection of Soft Tissue,” issued Aug. 14, 2001; U.S. Pat. No. 6,231,522, entitled “Biopsy Instrument with Breakable Sample Segments,” issued May 15, 2001; U.S. Pat. No. 6,228,055, entitled “Devices for Marking and Defining Particular Locations in Body Tissue,” issued May 8, 2001; U.S. Pat. No. 6,120,462, entitled “Control Method for an Automated Surgical Biopsy Device,” issued Sep. 19, 2000; U.S. Pat. No. 6,086,544, entitled “Control Apparatus for an Automated Surgical Biopsy Device,” issued Jul. 11, 2000; U.S. Pat. No. 6,077,230, entitled “Biopsy Instrument with Removable Extractor,” issued Jun. 20, 2000; U.S. Pat. No. 6,017,316, entitled “Vacuum Control System and Method for Automated Biopsy Device,” issued Jan. 25, 2000; U.S. Pat. No. 6,007,497, entitled “Surgical Biopsy Device,” issued Dec. 28, 1999; U.S. Pat. No. 5,980,469, entitled “Method and Apparatus for Automated Biopsy and Collection of Soft Tissue,” issued Nov. 9, 1999; U.S. Pat. No. 5,964,716, entitled “Method of Use for a Multi-Port Biopsy Instrument,” issued Oct. 12, 1999; U.S. Pat. No. 5,928,164, entitled “Apparatus for Automated Biopsy and Collection of Soft Tissue,” issued Jul. 27, 1999; U.S. Pat. No. 5,775,333, entitled “Apparatus for Automated Biopsy and Collection of Soft Tissue,” issued Jul. 7, 1998; U.S. Pat. No. 5,769,086, entitled “Control System and Method for Automated Biopsy Device,” issued Jun. 23, 1998; U.S. Pat. No. 5,649,547, entitled “Methods and Devices for Automated Biopsy and Collection of Soft Tissue,” issued Jul. 22, 1997; U.S. Pat. No. 5,526,822, entitled “Method and Apparatus for Automated Biopsy and Collection of Soft Tissue,” issued Jun. 18, 1996; U.S. Pub. No. 2008/0214955, entitled “Presentation of Biopsy Sample by Biopsy Device,” published Sep. 4, 2008; U.S. Pub. No. 2007/0255168, entitled “Grid and Rotatable Cube Guide Localization Fixture for Biopsy Device,” published Nov. 1, 2007; U.S. Pub. No. 2007/0118048, entitled “Remote Thumbwheel for a Surgical Biopsy Device,” published May 24, 2007; U.S. Pub. No. 2005/0283069, entitled “MRI Biopsy Device Localization Fixture,” published Dec. 22, 2005; U.S. Pub. No. 2003/0199753, entitled “MRI Compatible Biopsy Device with Detachable Probe,” published Oct. 23, 2003; U.S. Pub. No. 2003/0109803, entitled “MRI Compatible Surgical Biopsy Device,” published Jun. 12, 2003; U.S. Pub. No. 2008/0221480, entitled “Biopsy Sample Storage,” published Sep. 11, 2008; and U.S. Pub. No. 2008/0146962, entitled “Biopsy System with Vacuum Control Module,” published Jun. 19, 2008. The disclosure of each of the above-cited U.S. patents and U.S. patent application Publications is incorporated by reference herein.

Some biopsy systems may provide an apparatus to guide a probe and/or other components of a biopsy device to a desired biopsy site. In some such biopsy systems, a guide cube and positioning grid plate may be used. The guide cube may be selectively located within an opening in the grid plate. The guide cube may include guide holes to receive a portion of the probe and/or other components, for example a needle, cannula, obturator, or combinations of these or other components. With the guide cube inserted in the grid plate, the probe or other components can be guided through a selected guide hole of the guide cube to arrive at a desired biopsy site. The desired biopsy site may or may not have been identified and/or targeted by one or more of the guidance approaches mentioned above. In some situations, it might be desirable to provide a guide cube with features that improve a guide cube's use with one or more positioning grid plates. Merely exemplary biopsy device guides are disclosed in U.S. patent application Ser. No. 12/485,119, entitled “Biopsy Targeting Cube with Elastomeric Edges,” filed Jun. 16, 2009; U.S. patent application Ser. No. 12/485,138, entitled “Biopsy Targeting Cube with Elastomeric Body,” filed Jun. 16, 2009; U.S. patent application Ser. No. 12/485,168, entitled “Biopsy Targeting Cube with Malleable Members,” filed Jun. 16, 2009; U.S. patent application Ser. No. 12/485,278, entitled “Biopsy Targeting Cube with Angled Interface,” filed Jun. 16, 2009; and U.S. patent application Ser. No. 12/485,318, entitled “Biopsy Targeting Cube with Living Hinges,” filed Jun. 16, 2009. The disclosure of each of the above-cited U.S. patent applications is incorporated by reference herein.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an exemplary biopsy system including a control module remotely coupled to a biopsy device, and including a localization assembly;

FIG. 2 depicts a perspective view of a breast coil of the localization assembly of FIG. 1;

FIG. 3 depicts a perspective view of the biopsy device inserted through the guide cube of the localization assembly of FIG. 1;

FIG. 4 depicts a perspective view of the obturator and cannula of the biopsy system of FIG. 1;

FIG. 5 depicts an exploded perspective view of the obturator and cannula of FIG. 4;

FIG. 6 depicts a perspective view of the guide cube inserted into the grid plate of the localization assembly of FIG. 1;

FIG. 7 depicts a perspective view of the obturator and cannula of FIG. 4 with a depth stop device of FIG. 1 inserted through the guide cube and grid plate of FIG. 6;

FIG. 8 depicts a perspective view of the guide cube of the biopsy system of FIG. 1;

FIG. 9 depicts a diagram of nine guide positions achievable by rotating the guide cube of FIG. 8;

FIG. 10 depicts a perspective view of another guide cube for the biopsy system of FIG. 1, with a self-grounding feature;

FIG. 11 depicts a perspective view of the obturator and cannula of FIG. 1 inserted into one of two guide cubes of FIG. 10 inserted into the grid plate of FIG. 1;

FIG. 12 depicts a perspective view of another exemplary guide cube, having an open top and bottom with another self-grounding feature;

FIG. 13 depicts a rear perspective view of another exemplary guide cube, with another self-grounding feature;

FIG. 14 depicts a front perspective view of the guide cube of FIG. 13;

FIG. 15 depicts a right side view of the guide cube of FIG. 13 with angled, parallel guide holes depicted in phantom;

FIG. 16 depicts a perspective view of an exemplary alternative guide cube, with retention wipers;

FIG. 17 depicts a top plan view of the guide cube of FIG. 16;

FIG. 18 depicts a partial cross-sectional perspective view of the guide cube of FIG. 16; and

FIG. 19 depicts a side elevational view of the guide cube of FIG. 16 inserted in the grid plate of FIG. 6, with the grid plate shown in cross-section.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

As shown in the figures, an exemplary magnetic resonance imaging (MRI or MR imaging) compatible biopsy system may include a control module (12), localization assembly (15), and biopsy device (14). In particular, localization assembly (15) is configured to localize a patient's breast and guide needle (90) of biopsy device (14) to a targeted area within the patient's breast; while control module (12) is operable to control biopsy device (14) after needle (90) has been introduced to the target site. These components and their sub-components will be discussed further below. In addition, guide cubes for use with various localization assemblies will be discussed. While this disclosure may reference the biopsy system as compatible with MRI and MRI equipment and devices, it should be appreciated that other imaging techniques and equipment and devices may be used with the components described below, including but not limited to stereotactic, ultrasound, PEM, BSGI, and/or other imaging techniques and equipment.

### I. Control Module

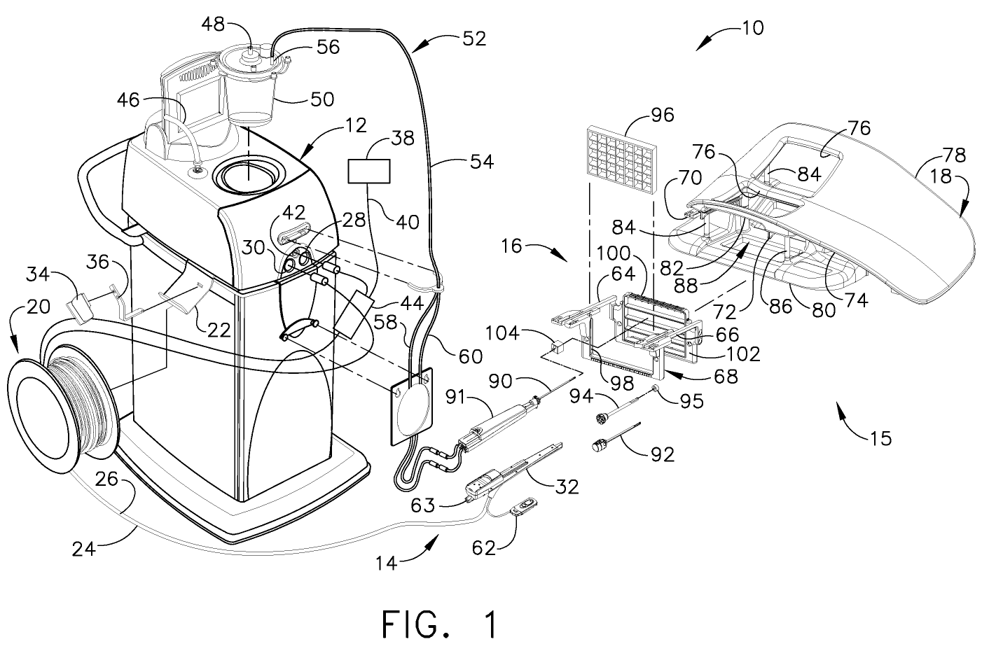

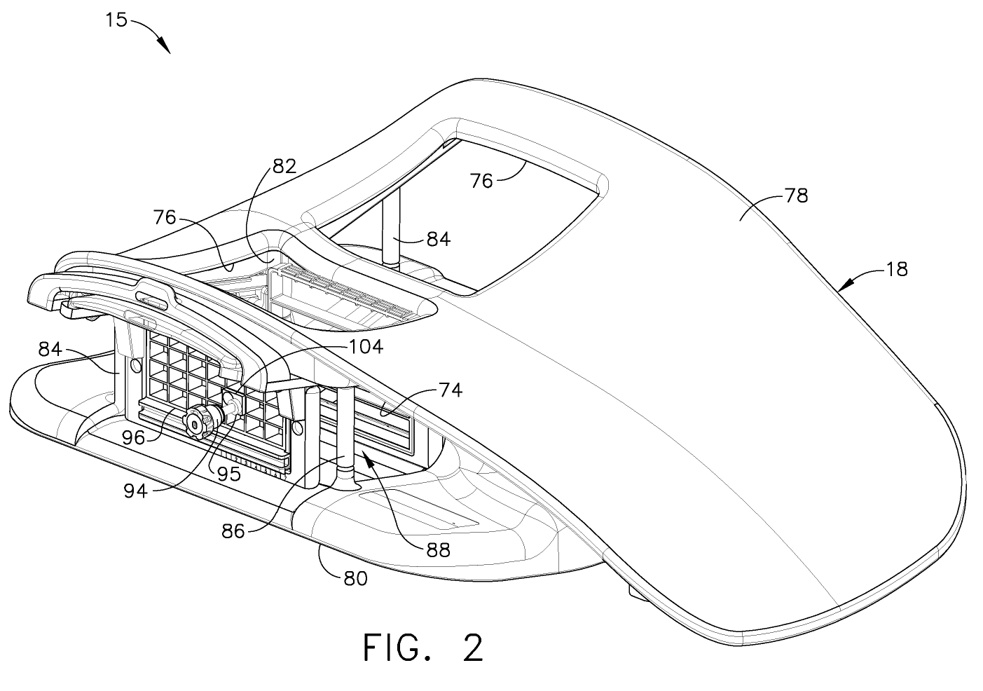

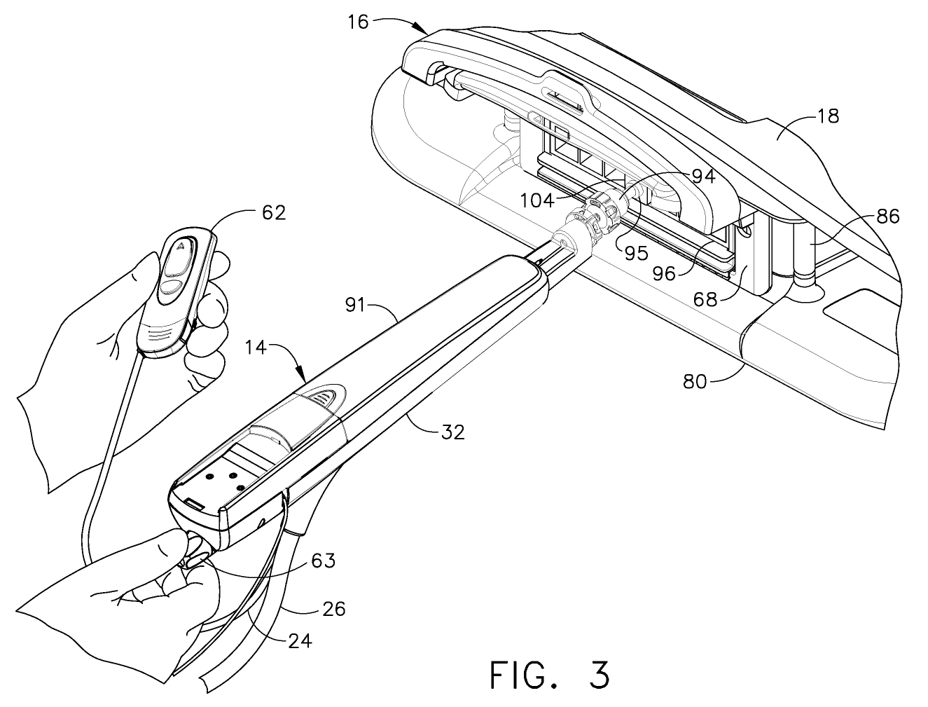

In FIGS. 1-3, MRI compatible biopsy system (10) has control module (12) that may be placed outside of a shielded room containing an MRI machine (not shown) or at least spaced away to mitigate detrimental interaction with its strong magnetic field and/or sensitive radio frequency (RF) signal detection antennas. As described in U.S. Pat. No. 6,752,768, which is hereby incorporated by reference in its entirety, a range of preprogrammed functionality may be incorporated into control module (12) to assist in taking tissue samples. Control module (12) controls and powers biopsy device (14) that is used with localization assembly (15). Biopsy device (14) is positioned and guided by localization fixture (16) attached to breast coil (18) that may be placed upon a gantry (not shown) of a MRI or other imaging machine.

In the present example, control module (12) is mechanically, electrically, and pneumatically coupled to biopsy device (14) so that components may be segregated that need to be spaced away from the strong magnetic field and the sensitive RF receiving components of a MRI machine. Cable management spool (20) is placed upon cable management attachment saddle (22) that projects from a side of control module (12). Wound upon cable management spool (20) is paired electrical cable (24) and mechanical cable (26) for communicating control signals and cutter rotation/advancement motions respectively. In particular, electrical and mechanical cables (24, 26) each have one end connected to respective electrical and mechanical ports (28, 30) in control module (12) and another end connected to holster portion (32) of biopsy device (14). Docking cup (34), which may hold holster portion (32) when not in use, is hooked to control module (12) by docking station mounting bracket (36). It should be understood that such components described above as being associated with control module (12) are merely optional.

Interface lock box (38) mounted to a wall provides tether (40) to lockout port (42) on control module (12). Tether (40) is uniquely terminated and of short length to preclude inadvertent positioning of control module (12) too close to a MRI machine or other machine In-line enclosure (44) may register tether (40), electrical cable (24) and mechanical cable (26) to their respective ports (42, 28, 30) on control module (12).

Vacuum assist is provided by first vacuum line (46) that connects between control module (12) and outlet port (48) of vacuum canister (50) that catches liquid and solid debris. Tubing kit (52) completes the pneumatic communication between control module (12) and biopsy device (14). In particular, second vacuum line (54) is connected to inlet port (56) of vacuum canister (50). Second vacuum line (54) divides into two vacuum lines (58, 60) that are attached to biopsy device (14). With biopsy device (14) installed in holster portion (32), control module (12) performs a functional check. Saline may be manually injected into biopsy device (14) or otherwise introduced to biopsy device (14), such as to serve as a lubricant and to assist in achieving a vacuum seal and/or for other purposes. Control module (12) actuates a cutter mechanism (not shown) in biopsy device (14), monitoring full travel of a cutter in biopsy device (14) in the present example. Binding in mechanical cable (26) or within biopsy device (14) may optionally monitored with reference to motor force exerted to turn mechanical cable (26) and/or an amount of twist in mechanical cable (26) sensed in comparing rotary speed or position at each end of mechanical cable (26).

Remote keypad (62), which is detachable from holster portion (32), communicates via electrical cable (24) to control panel (12) to enhance clinician control of biopsy device (14) in the present example, especially when controls that would otherwise be on biopsy device (14) itself are not readily accessible after insertion into localization fixture (16) and/or placement of control module (12) is inconveniently remote (e.g., 30 feet away). However, as with other components described herein, remote keypad (62) is merely optional, and may be modified, substituted, supplemented, or omitted as desired. In the present example, aft end thumbwheel (63) on holster portion (32) is also readily accessible after insertion to rotate the side from which a tissue sample is to be taken.

Of course, the above-described control module (12) is merely one example. Any other suitable type of control module (12) and associated components may be used. By way of example only, control module (12) may instead be configured and operable in accordance with the teachings of U.S. Pub. No. 2008/0228103, entitled “Vacuum Timing Algorithm for Biopsy Device,” published Sep. 18, 2008, the disclosure of which is incorporated by reference herein. As another merely illustrative example, control module (12) may instead be configured and operable in accordance with the teachings of U.S. patent application Ser. No. 12/337,814, entitled “Control Module Interface for MRI Biopsy Device,” filed Dec. 18, 2008, the disclosure of which is incorporated by reference herein. Alternatively, control module (12) may have any other suitable components, features, configurations, functionalities, operability, etc. Other suitable variations of control module (12) and associated components will be apparent to those of ordinary skill in the art in view of the teachings herein.

### II. Localization Assembly

Localization assembly (15) of the present example comprises breast coil (18) and localization fixture (16). These components of localization assembly (15) are described further below.

Left and right parallel upper guides (64, 66) of localization framework (68) are laterally adjustably received respectively within left and right parallel upper tracks (70, 72) attached to under side (74) and to each side of a selected breast aperture (76) formed in patient support platform (78) of breast coil (18). Base (80) of breast coil (18) is connected by centerline pillars (82) that are attached to patient support platform (78) between breast apertures (76). Also, a pair of outer vertical support pillars (84, 86) on each side spaced about a respective breast aperture (76) respectively define lateral recess (88) within which localization fixture (16) resides.

It should be appreciated that the patient's breasts hang pendulously respectively into breast apertures (76) within lateral recesses (88) in the present example. For convenience, herein a convention is used for locating a suspicious lesion by Cartesian coordinates within breast tissue referenced to localization fixture (16) and to thereafter selectively position an instrument, such as needle (90) of probe (91) that is engaged to holster portion (32) to form biopsy device (14). Of course, any other type of coordinate system or targeting techniques may be used. To enhance hands-off use of biopsy system (10), especially for repeated re-imaging within the narrow confines of a closed bore MRI machine, biopsy system (10) may also guide obturator (92) encompassed by cannula (94). Depth of insertion is controlled by depth stop device (95) longitudinally positioned on either needle (90) or cannula (94). Alternatively, depth of insertion may be controlled in any other suitable fashion.

This guidance is specifically provided by a lateral fence in the present example, depicted as grid plate (96), which is received within laterally adjustable outer three-sided plate bracket (98) attached below left and right parallel upper guides (64, 66). Similarly, a medial fence with respect to a medial plane of the chest of the patient, depicted as medial plate (100), is received within inner three-sided plate bracket (102) attached below left and right parallel upper guides (64, 66) close to centerline pillars (82) when installed in breast coil (18). To further refine the insertion point of the instrument (e.g., needle (90) of probe (91), obturator/cannula (92, 94), etc.), guide cube (104) may be inserted into grid plate (96).

In the present example, the selected breast is compressed along an inner (medial) side by medial plate (100) and on an outer (lateral) side of the breast by grid plate (96), the latter defining an X-Y plane. The X-axis is vertical (sagittal) with respect to a standing patient and corresponds to a left-to-right axis as viewed by a clinician facing the externally exposed portion of localization fixture (16). Perpendicular to this X-Y plane extending toward the medial side of the breast is the Z-axis, which typically corresponds to the orientation and depth of insertion of needle (90) or obturator/cannula (92, 94) of biopsy device (14). For clarity, the term Z-axis may be used interchangeably with “axis of penetration”, although the latter may or may not be orthogonal to the spatial coordinates used to locate an insertion point on the patient. Versions of localization fixture (16) described herein allow a non-orthogonal axis of penetration to the X-Y axis to a lesion at a convenient or clinically beneficial angle.

It should be understood that the above-described localization assembly (15) is merely one example. Any other suitable type of localization assembly (15) may be used, including but not limited to localization assemblies (15) that use a breast coil (18) and/or localization fixture (16) different from those described above. Other suitable components, features, configurations, functionalities, operability, etc. for a localization assembly (15) will be apparent to those of ordinary skill in the art in view of the teachings herein.

### III. Biopsy Device

As shown in FIG. 1, one version of biopsy device (14) may comprise holster portion (32) and probe (91). Exemplary holster portion (32) was discussed previously in the above section addressing control module (12). The following paragraphs will discuss probe (91) and associated components and devices in further detail.

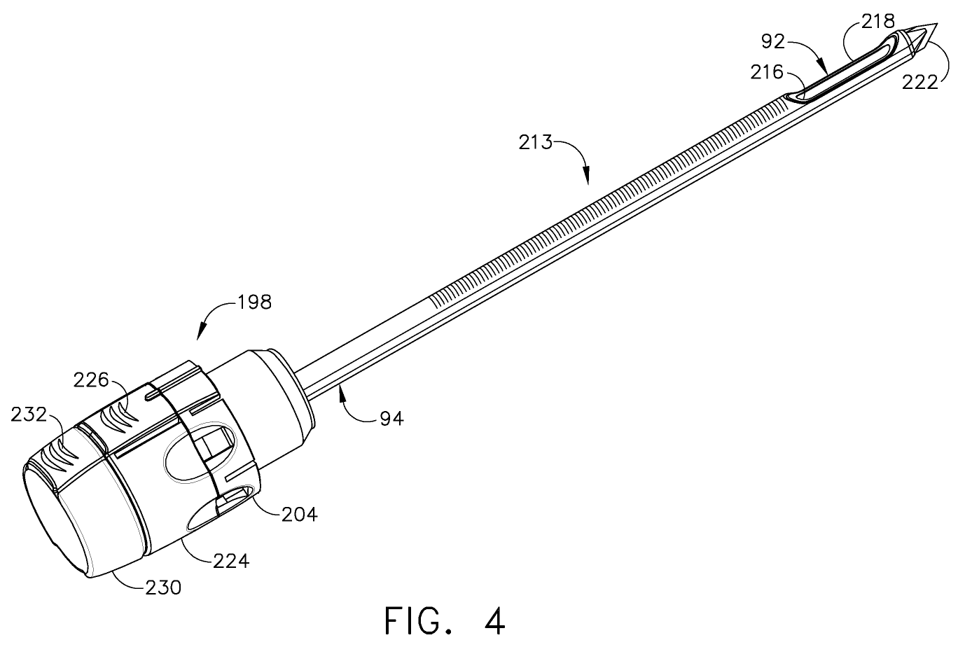

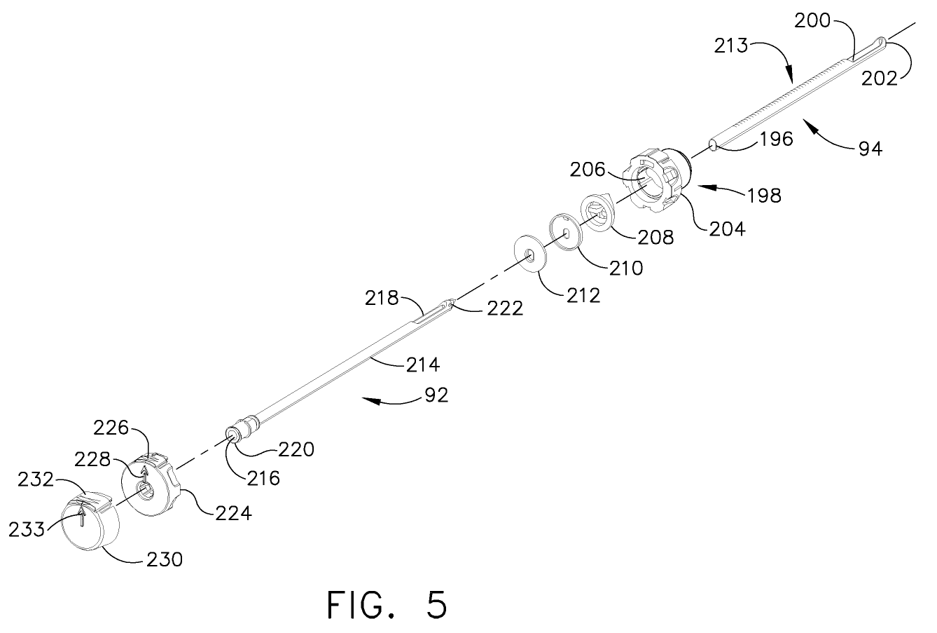

In the present example, cannula (94) and obturator (92) are associated with probe (91). In particular, and as shown in FIGS. 4, 5, and 7, obturator (92) is slid into cannula (94) and the combination is guided through guide cube (104) to the biopsy site within the breast tissue. Obturator (92) is then withdrawn from cannula (94), then needle (90) of probe (91) is inserted in cannula (94), and then biopsy device (14) is operated to acquire one or more tissue samples from the breast via needle (90).

Cannula (94) of the present example is proximally attached to cylindrical hub (198) and cannula (94) includes lumen (196) and lateral aperture (200) proximate to open distal end (202). Cylindrical hub (198) has exteriorly presented thumbwheel (204) for rotating lateral aperture (200). Cylindrical hub (198) has interior recess (206) that encompasses duckbill seal (208), wiper seal (210) and seal retainer (212) to provide a fluid seal when lumen (196) is empty and for sealing to inserted obturator (92). Longitudinally spaced measurement indicia (213) along an outer surface of cannula (94) visually, and perhaps physically, provide a means to locate depth stop device (95) of FIG. 1.

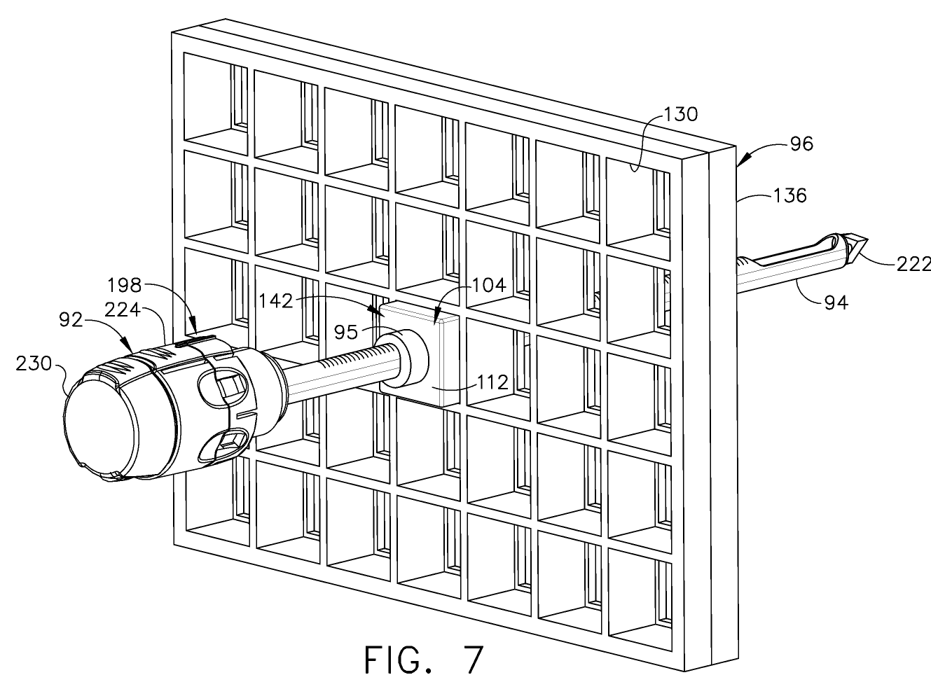

Obturator (92) of the present example incorporates a number of components with corresponding features. Hollow shaft (214) includes fluid lumen (216) that communicates between imageable side notch (218) and proximal port (220). Hollow shaft (214) is longitudinally sized to extend, when fully engaged with cannula (94), piercing tip (222) out of distal end (202) of cannula (94). Obturator thumbwheel cap (224) encompasses proximal port (220) and includes locking feature (226), which includes visible angle indicator (228), that engages cannula thumbwheel (204) to ensure that imageable side notch (218) is registered to lateral aperture (200) in cannula (94). Obturator seal cap (230) may be engaged proximally into obturator thumbwheel cap (224) to close fluid lumen (216). Obturator seal cap (230) of the present example includes locking or locating feature (232) that includes visible angle indicator (233) that corresponds with visible angle indicator (228) on obturator thumbwheel cap (224), which may be fashioned from either a rigid, soft, or elastomeric material. In FIG. 7, guide cube (104) has guided obturator (92) and cannula (94) through grid plate (96).

While obturator (92) of the present example is hollow, it should be understood that obturator (92) may alternatively have a substantially solid interior, such that obturator (92) does not define an interior lumen. In addition, obturator (92) may lack side notch (218) in some versions. Other suitable components, features, configurations, functionalities, operability, etc. for an obturator (92) will be apparent to those of ordinary skill in the art in view of the teachings herein. Likewise, cannula (94) may be varied in a number of ways. For instance, in some other versions, cannula (94) has a closed distal end (202). As another merely illustrative example, cannula (94) may have a closed piercing tip (222) instead of obturator (92) having piercing tip (222). In some such versions, obturator (92) may simply have a blunt distal end; or the distal end of obturator (92) may have any other suitable structures, features, or configurations. Other suitable components, features, configurations, functionalities, operability, etc. for a cannula (94) will be apparent to those of ordinary skill in the art in view of the teachings herein. Furthermore, in some versions, one or both of obturator (92) or cannula (94) may be omitted altogether. For instance, needle (90) of probe (91) may be directly inserted into a guide cube (104), without being inserted into guide cube (104) via cannula (94).

Another component that may be used with probe (91) (or needle (90)) is depth stop (95). Depth stop may be of any suitable configuration that is operable to prevent cannula (94) and obturator (92) (or needle (90)) from being inserted further than desired. For instance, depth stop (95) may be positioned on the exterior of cannula (94) (or needle (90)), and may be configured to restrict the extent to which cannula (94) is inserted into a guide cube. It should be understood that such restriction by depth stop (95) may further provide a limit on the depth to which the combination of cannula (94) and obturator (92) (or needle (90)) may be inserted into the patient's breast. Furthermore, it should be understood that such restriction may establish the depth within the patient's breast at which biopsy device (14) acquires one or more tissue samples after obturator (92) has been withdrawn from cannula (94) and needle (90) has been inserted in cannula (94). Exemplary depth stops (95) that may be used with biopsy system (10) are described in U.S. Pub. No. 2007/0255168, entitled “Grid and Rotatable Cube Guide Localization Fixture for Biopsy Device,” published Nov. 1, 2007, and incorporated by reference herein as mentioned previously.

In the present example, and as noted above, biopsy device (14) includes a needle (90) that may be inserted into cannula (94) after the combination of cannula (94) and obturator (92) has been inserted to a desired location within a patient's breast and after obturator (92) has been removed from cannula (94). Needle (90) of the present example comprises a lateral aperture (not shown) that is configured to substantially align with lateral aperture (200) of cannula (94) when needle (90) is inserted into lumen (196) of cannula (94). Probe (91) of the present example further comprises a rotating and translating cutter (not shown), which is driven by components in holster (32), and which is operable to sever tissue protruding through lateral aperture (200) of cannula (94) and the lateral aperture of needle (90). Severed tissue samples may be retrieved from biopsy device (14) in any suitable fashion.

By way of example only, biopsy device (14) may be configured and operable in accordance with the teachings of U.S. Pub. No. 2008/0228103, entitled “Vacuum Timing Algorithm For Biopsy Device,” published Sep. 18, 2008, the disclosure of which is incorporated by reference herein. As another merely illustrative example, biopsy device (14) may be configured and operable in accordance with the teachings of U.S. patent application Ser. No. 12/337,874, entitled “Mechanical Tissue Sample Holder Indexing Device,” filed Dec. 18, 2008, the disclosure of which is incorporated by reference herein. As another merely illustrative example, biopsy device (14) may be configured and operable in accordance with the teachings of U.S. patent application Ser. No. 12/337,674, entitled “Biopsy Device with Sliding Cutter Cover,” filed Dec. 18, 2008, the disclosure of which is incorporated by reference herein. By way of example only, cannula (94) may be replaced with any of the detachable needles described in U.S. patent application Ser. No. 12/337,674, entitled “Biopsy Device with Sliding Cutter Cover.” As another merely illustrative example, biopsy device (14) may be configured and operable in accordance with the teachings of U.S. patent application Ser. No. 12/337,911, entitled “Biopsy Device with Discrete Tissue Chambers,” filed Dec. 18, 2008, the disclosure of which is incorporated by reference herein. As another merely illustrative example, biopsy device (14) may be configured and operable in accordance with the teachings of U.S. patent application Ser. No. 12/337,942, entitled “Biopsy Device with Central Thumbwheel,” filed Dec. 18, 2008, the disclosure of which is incorporated by reference herein. Alternatively, biopsy device (14) may have any other suitable components, features, configurations, functionalities, operability, etc. Other suitable variations of biopsy device (14) and associated components will be apparent to those of ordinary skill in the art in view of the teachings herein

### IV. Guide Cubes

Guide cubes described below are generally adapted for use with a localization assembly (15) as described above. Numerous features of merely exemplary guide cubes will be discussed in the paragraphs that follow.

### A. Guide Cubes Generally

In some versions, guide cubes may comprise a body defined by one or more edges and faces. The body may include one or more guide holes or other types of passages that extend between faces of the guide cube and that may be used to guide an instrument such as a biopsy device (14) or a portion of a biopsy device (14) (e.g., needle (90) of biopsy device (14), a combination of cannula (94) and obturator (92), etc.). Guide cubes may be rotatable about one, two, or three axes to position the one or more guide holes or passages of the guide cube into a desired position.

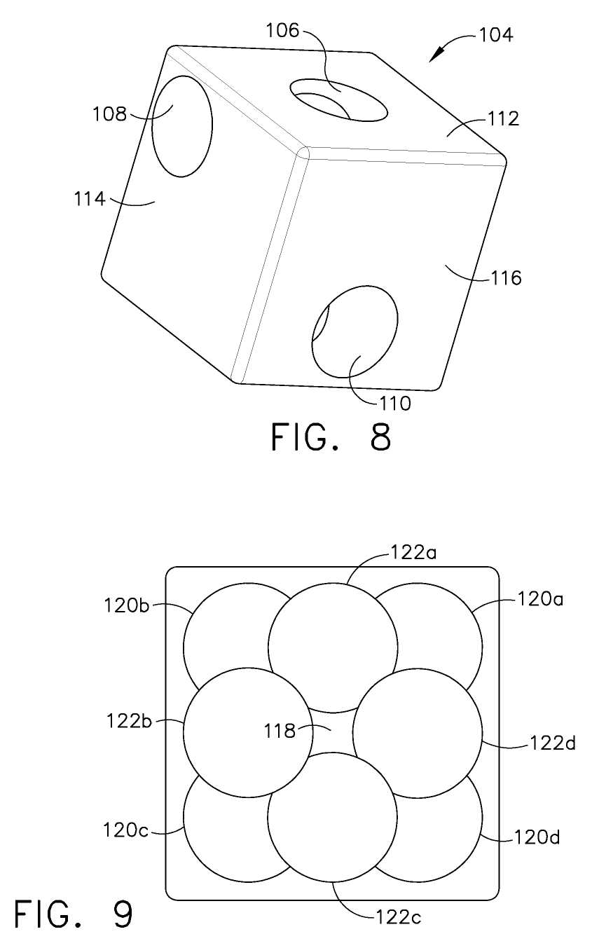

Referring now to FIG. 8, guide cube (104), includes central guide hole (106), corner guide hole (108), and off-center guide hole (110) that pass orthogonally to one another between respective opposite pairs of faces (112, 114, 116). By selectively rotating guide cube (104) in two axes, one pair of faces (112, 114, 116) may be proximally aligned to an unturned position and then the selected proximal face (112, 114, 116) optionally rotated a quarter turn, half turn, or three-quarter turn. Thereby, one of nine guide positions (118, 120a-120d, 122a-122d) may be proximally exposed as depicted in FIG. 9. More specifically, central guide hole (106) may provide for guide position (118), corner guide hole (108) may provide for guide positions (120a-120d), and off-center guide hole (110) may provide for guide positions (122a-122d).

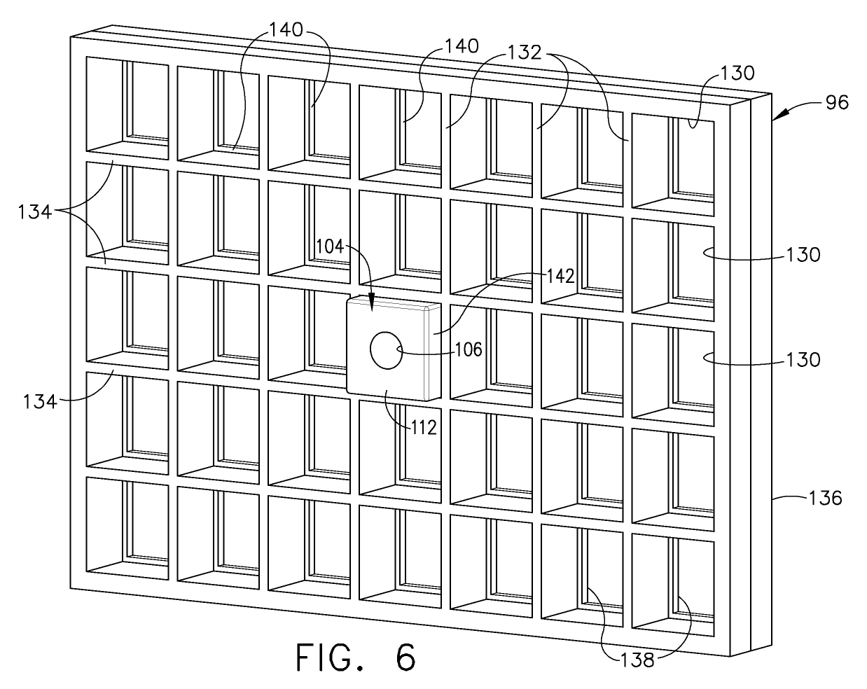

In FIG. 6, two-axis rotatable guide cube (104) is sized for insertion from a proximal side into one of a plurality of square recesses (130) in grid plate (96), which are formed by intersecting vertical bars (132) and horizontal bars (134). Guide cube (104) is prevented from passing through grid plate (96) by backing substrate (136) attached to a front face of grid plate (96). Backing substrate (136) includes respective square opening (138) centered within each square recess (130), forming lip (140) sufficient to capture the front face of guide cube (104), but not so large as to obstruct guide holes (104, 106, 108). The depth of square recesses (130) is less than guide cube (104), thereby exposing a proximal portion (142) of guide cube (104) for seizing and extraction from grid plate (96). It will be appreciated by those of ordinary skill in the art based on the teachings herein that backing substrate (136) of grid plate (96) may be omitted altogether in some versions. In some such versions without backing substrate (136) other features of a guide cube, as will be discussed in more detail below, may be used to securely and removably fit a guide cube within a grid plate. However, such other features may also be used in combination with a grid plate having backing substrate (136), such as grid plate (96), instead of partially or wholly omitting backing substrate (136).

### B. Self-Grounding Guide Cubes

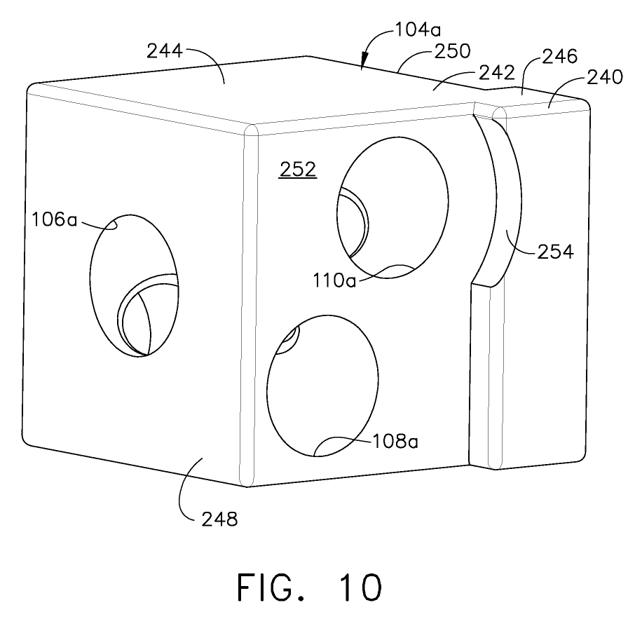

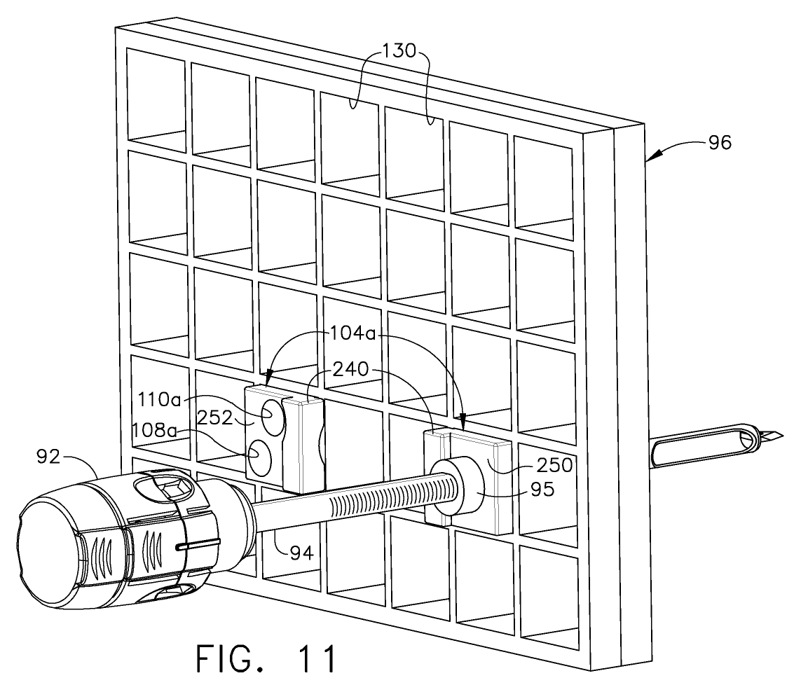

In FIG. 10, guide cube (104a) has self-grounding by means of added rectangular prism (240) which has a shared edge with cubic portion (242) of guide cube (104a). When viewed orthogonally to the shared cube edge, larger square face (244) of cubic portion (242) overlaps with smaller square face (246) of rectangular prism (240). As shown in FIG. 11, rectangular prism (240) allows proximal exposure of one of two adjacent faces (250, 252) of guide cube (104a) and then turning each to one of four quarter-turn rotational positions. In the illustrative version, first face (250) has central guide hole (106a) and second face (252) has corner guide hole (108a), and off-center guide hole (110a). Radial recess (254) is formed in rectangular prism (240) to allow grounding of depth stop device (95) against face (252) when off-center guide hole (110a) is used.

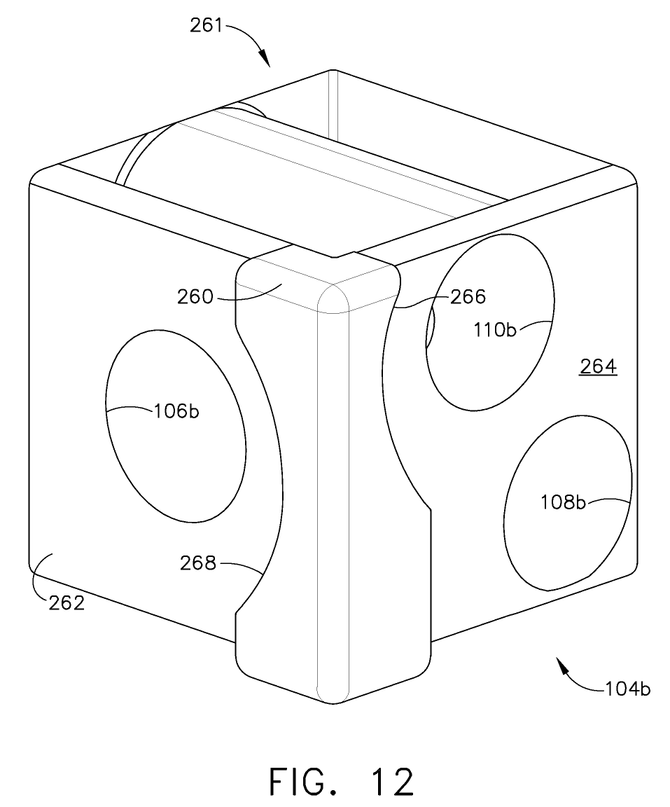

In FIG. 12, guide cube (104b) has self-grounding by means of added rectangular prism (260) that protrudes from two faces (262, 264) of guide cube (104b). Rectangular prism (260) allows proximal exposure of one of two adjacent faces (262, 264) of guide cube (104b) and then turning each to one of four quarter-turn rotational positions. In the illustrative version, first face (262) has central guide hole (106b) and second face (264) has corner guide hole (108b) and off-center guide hole (110b). First radial recess (266) is formed in rectangular prism (260) to allow grounding of depth stop device (95) against face (264) when off-center guide hole (110b) is used. Second radial recess (268) is formed in rectangular prism (260) to allow grounding of depth stop device (95) against face (262) when central guide hole (106b) is used. As discussed in greater detail below, guide cube (104b) may have open top (261) and/or an open bottom (not shown) defined by the faces of guide cube (104b) as depicted in the illustrated version.

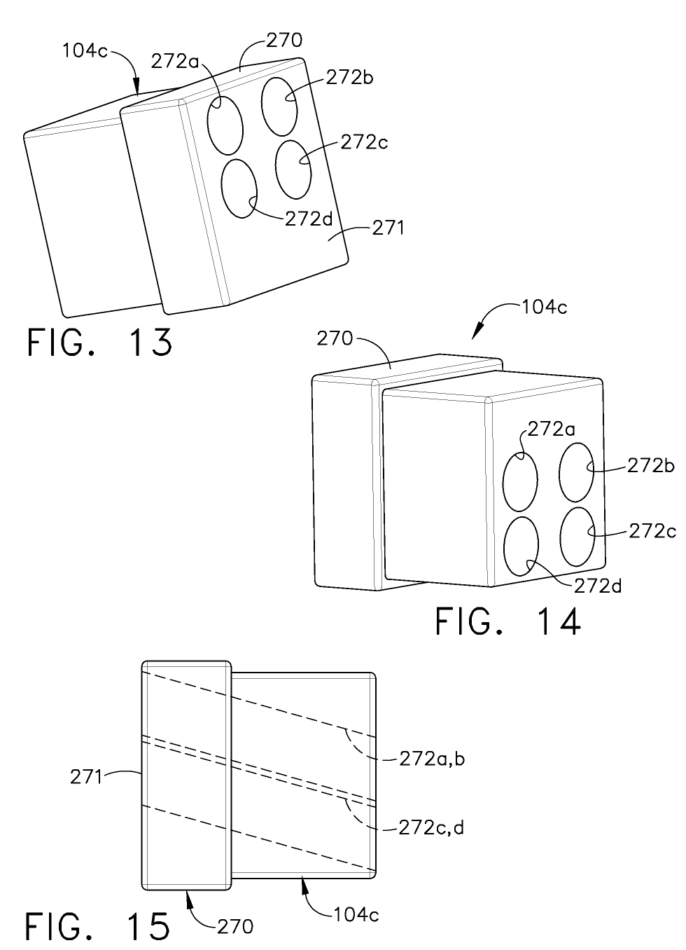

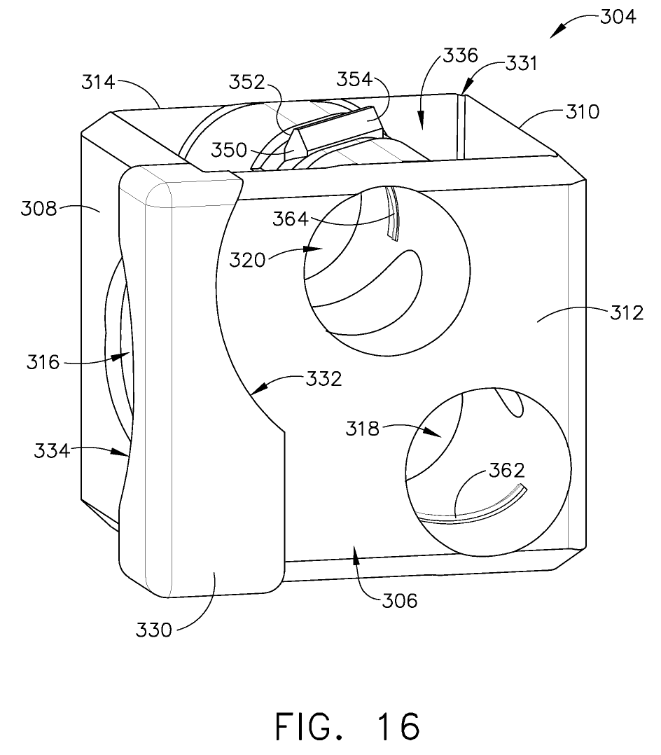

In FIGS. 13-15, guide cube (104c) has proximal enlarged hat portion (270) about proximal face (271) that grounds against selected square recess (130), such as in grid plate (96), and allows rotation about one axis to one of four quarter-turn positions. Four angled guide holes (272a, 272b, 272c, 272d) allow accessing not only an increased number of insertion points within selected square recess (130) but also a desired angle of penetration rather than being constrained to a perpendicular insertion. It will be appreciated based on the teachings herein that while angled guide holes may be used in some versions, orthogonal guide holes may be used instead of or in addition to angled guide holes in other versions.

C. Guide Cube with Wiper

In some versions of guide devices, the guide device may include features that assist in securing the guide device within an aperture of a grid plate. Such features may be configured to secure the guide device from movement in a proximal direction, distal direction, lateral direction, or combinations of these or other directions. For instance, such features may substantially retain the guide device by providing restriction on or resistance to movement of the guide device relative to the grid plate (96) upon sufficient engagement between the guide device and grid plate (96). In some versions of guide devices, the guide devices may further include features that assist in securing an instrument, such as a biopsy device (14) or a portion of a biopsy device (14) (e.g., needle (90) of biopsy device (14), a combination of cannula (94) and obturator (92), etc.), within a selected guide hole or passageway of the guide device. In some versions, such features may substantially retain the instrument or portion of the instrument by providing resistance to movement of the instrument in a proximal direction, distal direction, rotational direction, lateral direction, or combinations of these or other directions. The paragraphs that follow will describe merely exemplary versions of guide devices or modifications to guide devices that may include some of these optional features, among other features.

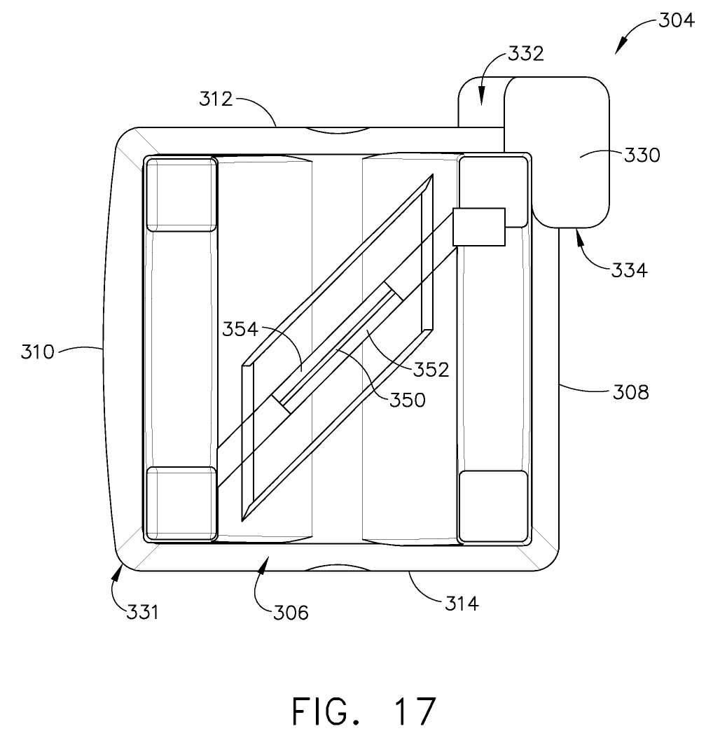

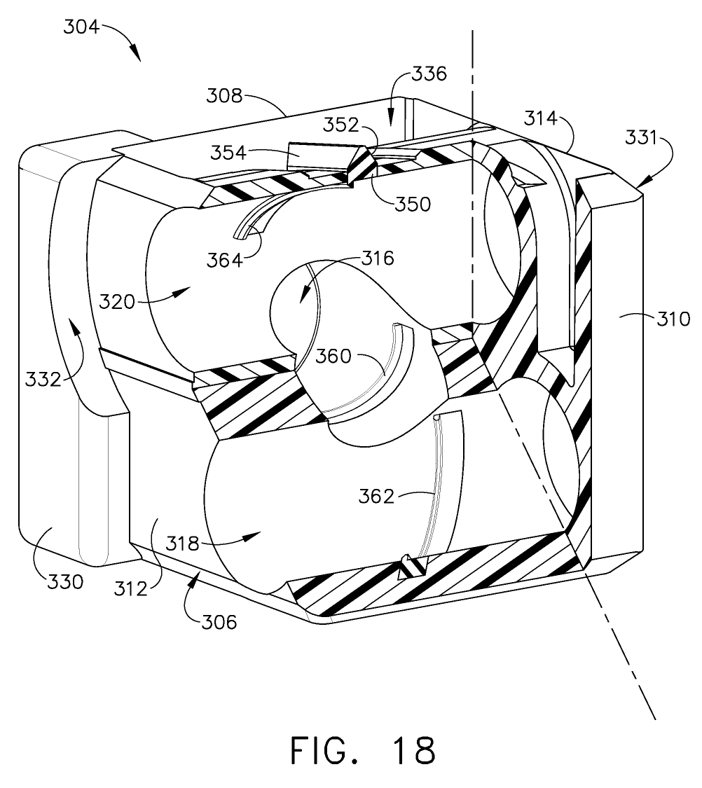

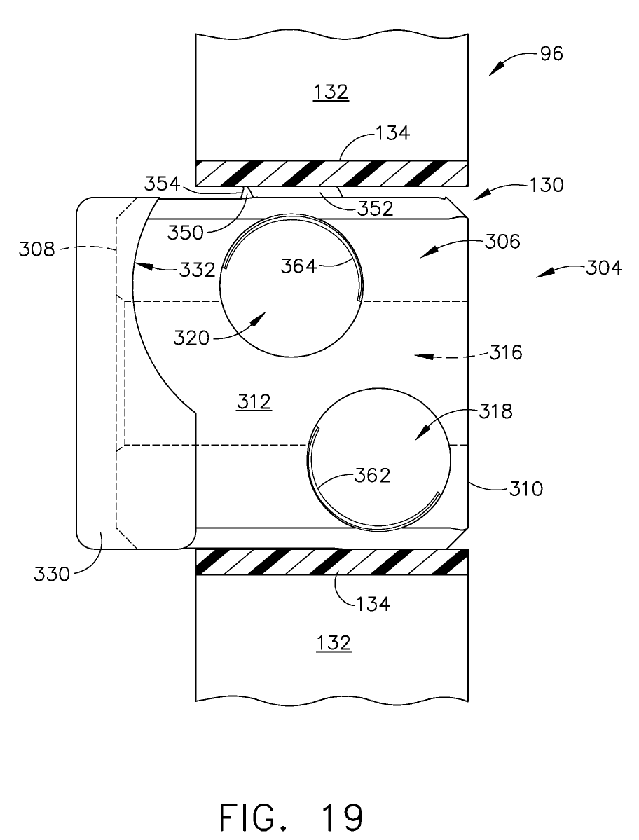

In FIGS. 16-18, an exemplary guide cube (304) includes body (306) defined by four faces (308, 310, 312, 314). Faces (308, 310, 312, 314) include two sets of opposing faces, as shown in the illustrated version where face (308) and face (310) are opposing and likewise face (312) and face (314) are opposing. Guide cube (304) has guide passageways (316, 318, 320) passing through guide cube (304). Guide passageways (316, 318, 320) have corresponding openings in a set of opposing faces thereby providing access via a passageway from one side of guide cube (304) to the other side. It should be appreciated that guide passageways (316, 318, 320) may be configured to share a common opening in a face in some versions. As shown in the illustrated version, faces (308, 310) include central guide passageway (316), while faces (312, 314) include corner guide passageway (318) and off-center guide passageway (320). However, it should be understood that faces (308, 310, 312, 314) may each have any suitable number of guide passageways in any suitable positioning or arrangements, and that any suitable number of passages may be provided through guide cube (304). As shown in FIG. 18, guide passageways (316, 318, 320) are sized and overlappingly arranged such that guide passageways (316, 318, 320) are in communication with each other. In the present example, despite this overlapping arrangement, guide passageways (316, 318, 320) are orthogonally oriented such that an instrument is insertable in only one guide passageway (316, 318, 320) at a time. However, some other versions may include overlapping guide passageways where an instrument may insertingly “cross over” from one guide passageway to another, such as to achieve a non-orthogonal angular orientation relative to the guide cube. It should also be understood that guide passageways (316, 318, 320) may simply not overlap or otherwise be in communication with each other in some versions.

Each face (308, 310, 312, 314) of guide cube (304) may be defined by edges. In such a configuration, it will be appreciated that some faces (308, 310, 312, 314) may share one or more common edges. It should be further appreciated that faces (308, 310, 312, 314) may be configured such that each does not share common edges, but rather the edges of adjacent faces abut one another forming the edges of guide cube (304). For instance, faces (308, 310, 312, 314) may be initially formed separately, such as by being formed as separate plates, with each plate having its own four edges, and with the separate plates being joined together to form guide cube (304), etc.). In some versions, at least a portion of edges of guide cube (304) may be comprised of or fitted with elastomeric material (not shown); while some other portions of guide cube (304) are formed of another material such as hard plastic. Alternatively, any other suitable material or materials may be used.

Guide cube (304) may further be rotatable about two axes with self-grounding by means of rectangular prism (330) that protrudes from two faces (308, 312) of guide cube (304). Rectangular prism (330) allows proximal exposure of one of two adjacent faces (308, 312) of guide cube (304) relative to grid plate (96). Thus, guide cube (304) may be selectively rotated about a first axis to selectively expose face (308) or face (312) proximally relative to grid plate (96). It should also be understood that, when a particular face (308, 312) has been selected and oriented for proximal exposure relative to grid plate (96), guide cube (304) may be rotated about a second axis to provide selected positioning of the presented guide passageway(s) (316, 318, 320). In other words, the proximally exposed face (308, 312) may be rotated to a selected one of four quarter-turn rotational positions. In the illustrative version, first radial recess (332) is formed in rectangular prism (330), providing clearance to allow grounding of depth stop device (95) against face (312) when off-center guide hole (320) is used. Second radial recess (334) is formed in rectangular prism (330), providing clearance to allow grounding of depth stop device (95) against face (308) when central guide hole (316) is used.

Guide cube (304) may have open top (336) and/or an open bottom (not shown) defined by faces (308, 310, 312, 314) of guide cube (304) as depicted in the illustrated version. Open top (336) and open bottom (not shown) may provide void volume within guide cube (304), and depending on the rigidity of the body of guide cube (304), the body of guide cube (304) may flex to some degree thereby permitting better fit within a grid plate (96) or more compatible fit within various grid plates. Alternatively, guide cube (304) may have a closed top and/or bottom. Similarly, aside from guide passageways (316, 318, 320), the interior of guide cube (304) may be substantially hollow or substantially solid, as desired.

Guide cube (304) of the present example further comprises an exterior retainer (350) and three interior retainers (360, 362, 364), all of which are configured as bendable or deformable wipers. Exterior retainer (350) is provided at the open top (336) of guide cube (304) in this example, though it should be understood that exterior retainer (350) may be provided at any other suitable location. In addition, while guide cube (304) of the present example includes just one exterior retainer (350), it should be understood that guide cube (304) may have zero, two, or more than two exterior retainers (350), as desired. Exterior retainer (350) is oriented such that it extends along an oblique, non-orthogonal plane that intersects rectangular prism (330) and the corner (331) that opposes rectangular prism (330). Accordingly, exterior retainer (350) will engage a portion of grid plate (96) at an angle of approximately 45 degrees regardless of whether guide cube (304) is inserted with face (314) first or with face (310) first. Of course, exterior retainer (350) may have any other suitable orientation. Exterior retainer (350) also has a tapered configuration in the present example, presenting angled faces (352, 354), though it should be understood that exterior retainer (350) may have any other suitable configuration.

As best seen in FIGS. 16 and 19, exterior retainer (350) extends upwardly past the upper edges of faces (308, 310, 312, 314). Thus, when guide cube (304) is inserted into an opening (130) of grid plate (96), exterior retainer (350) is configured to engage a portion of grid plate (96). FIG. 19 shows exterior retainer (350) engaging an upper horizontal bar (134) of grid plate (96). Of course, exterior retainer (350) may alternatively engage a lower horizontal bar (134) or either adjacent vertical bar (132), depending on the rotational orientation of guide cube (304) about the axis extending through the opening (130) of guide plate (96). As is also shown in FIG. 19, exterior retainer (350) deforms, bends, or folds rearwardly upon insertion of guide cube (304) in opening (130) of grid plate (96). In particular, exterior retainer (350) is bent or folded such that one angled face (352) contacts horizontal bar (134) of grid plate (96); while the other angled face (354) is bent away from horizontal bar (134). This bendability or deformability of exterior retainer (350) may permit guide cube (304) to be inserted into a selected opening (130) of grid plate (96) with relative ease. Conversely, this bendability or deformability of exterior retainer (350) may provide resistance to removal of guide cube (304) from the selected opening (130) of grid plate (96). For instance, and particularly when exterior retainer (350) is formed of an elastomeric material providing a relatively high coefficient of friction, a rearwardly bent exterior retainer (350) as shown in FIG. 19 may make proximal movement of the inserted guide cube (304) relatively more difficult than distal movement of the inserted guide cube (304).

It should therefore be understood that exterior retainer (350) may create a substantially secure interference between grid plate (96) and guide cube (304) without significantly increasing the force required to insert or remove guide cube (304) from grid plate (96). Accordingly, guide cube (304) of the present example may fit in various types of grid plates having grid openings or recesses of various sizes or configurations. It should also be understood that, in some settings, elastomeric exterior retainer (350) may provide sufficient friction with grid plate (96) to reduce the likelihood that guide cube (304) will undesirably fall out of grid plate (96). In addition, other suitable features, configurations, components, functionalities, operability, and variations of guide cube (304) will be apparent to those of ordinary skill in the art in view of the teachings herein. While exterior retainer (350) is shown and described as a feature providing resistance to withdrawal of guide cube (304) from grid plate (96) while not significantly providing resistance to insertion of guide cube (304) in grid plate (96), it should be understood that a variety of other components or features may be used to provide similar results. Similarly, it should be understood that guide exterior retainer (350) may be modified or varied in numerous ways, if not be omitted altogether. Various ways in which exterior retainer (350) may be modified, varied, substituted, or supplemented will be apparent to those of ordinary skill in the art in view of the teachings herein.

In the present example, interior retainer (360) is positioned within guide passageway (316); interior retainer (362) is positioned within guide passageway (318); and interior retainer (364) is positioned within guide passageway (320). As shown in FIG. 18, interior retainer (364) is formed with exterior retainer (350) as a single homogenous continuum of material in the present example, though it should be understood that interior retainer (364) and exterior retainer (350) may be formed as separate pieces and/or of different materials. Similarly, while all retainers (350, 360, 362, 364) are formed of the same elastomeric material in the present example, it should be understood that different retainers (350, 360, 362, 364) within a given guide cube (304) may be formed of different materials. Interior retainers (360, 362, 364) each present a circumferential “knife edge” configuration, similar to the tapered configuration of exterior retainer (350) of the present example. Of course, any other suitable configurations may be used.

In the present example, interior retainers (362, 364) extend along planes that are oblique relative to faces (308, 310, 312, 314) of guide cube (304). For instance, in some versions, interior retainers (362, 364) extend along a generally helical path in their respective guide passageways (318, 320). Furthermore, interior retainer (362) extends along a plane that is perpendicular to the plane along which interior retainer (364) extends. Interior retainer (360) extends along a plane that is transverse to the central axis of guide passageway (316) in some versions, though interior retainer (360) may alternatively extend along a generally helical path in guide passageway (316). In some settings, the generally helical orientation of interior retainers (360, 362, 364) may reduce the maximum force that is required to insert an instrument (e.g., cannula (94)) through the associated guide passageway (316, 318, 320). For instance, the inserted instrument may deflect only a small portion of interior retainer (360, 362, 364) as it is inserted into the associated guide passageway (316, 318, 320). By contrast, in some versions where interior retainers (360, 362, 364) are circular and/or extend about a path that is perpendicular to the longitudinal axis of the associated guide passageway (316, 318, 320), the leading edge of the inserted instrument may encounter the full circumference of the interior retainer (360, 362, 364) as the instrument is being inserted, which may provide relatively greater resistance to such insertion. It should be understood, however, that interior retainers (360, 362, 364) may extend at any other suitable orientations, as desired.

Also in the present example, interior retainers (360, 362, 364) extend circumferentially about 180 degrees within their respective guide passageways (316, 318, 320). It should be understood, though, that interior retainers (360, 362, 364) may alternatively extend circumferentially to any other suitable degree within their respective guide passageways (316, 318, 320). It should also be understood that use the term “circumferentially” is only intended to denote an angular distance about which the interior retainer (360, 362, 364) extends relative to the axis defined by the associated guide passageway (316, 318, 320). Use of the term “circumferentially” is not intended to imply that interior retainers (360, 362, 364) must extend only within a plane that is transverse to the axis defined by the associated guide passageway (316, 318, 320). While some versions of interior retainers (360, 362, 364) may in fact extend only within such a plane, other versions of interior retainers (360, 362, 364) may extend in a partially helical path or in some other orientation while still having a “circumferential” dimension to their extension.

Interior retainers (360, 362, 364) of the present example are operatively configured to assist in securing an instrument such as a biopsy device (14) or a portion of a biopsy device (14) (e.g., needle (90) of biopsy device (14), a combination of cannula (94) and obturator (92), etc.) within a selected guide passageway (316, 318, 320). Interior retainers (360, 362, 364) are comprised of an elastomeric material in some versions. In the present example, interior retainers (360, 362, 364) are configured such that the opening defined by the combination of interior retainer (360, 362, 364) and its corresponding guide passageway (316, 318, 320) is smaller in diameter than the diameter of the instrument, e.g. cannula (94), that is to be inserted in a selected guide passageway (316, 318, 320). When cannula (94) is inserted in a selected guide passageway (316, 318, 320), interior retainer (360, 362, 364) compresses, deforms, and/or folds over to provide for a secure fit. In other words, while interior retainer (360, 362, 364) permits distal insertion of cannula (94) or needle (90), etc., through a selected guide opening (316, 318, 320), friction between the inserted instrument and the elastomeric material of interior retainer (360, 362, 364) provides some resistance to proximal movement of the inserted instrument relative to guide passageway (316, 318, 320). In some versions, the securing force provided by interior retainer (360, 362, 364) is such that the compressed tissue of a patient will not displace cannula (94) proximally from guide passageway (316, 318, 320) during a biopsy procedure. It should also be understood that interior retainers (360, 362, 364) may bend or fold distally in response to distal inserting engagement by an instrument, in a manner similar to the proximal bending or folding of exterior retainer (350) described above, and that such a bent/folded configuration may further provide resistance against proximal withdrawal of the instrument.

It should also be understood that each guide passageway (316, 318, 320) may have more than one associated interior retainer (360, 362, 364). For instance, each guide passageway (316, 318, 320) may have two or more interior retainers (360, 362, 364) that are axially staggered along the length of guide passageway (316, 318, 320).

While interior retainers (360, 362, 364) are shown and described as a feature providing resistance to withdrawal of an inserted instrument from guide cube (304) while not significantly providing resistance to insertion of the instrument into guide cube (304), it should be understood that a variety of other components or features may be used to provide similar results. Similarly, it should be understood that interior retainers (360, 362, 364) may be modified or varied in numerous ways, if not be omitted altogether. Various ways in which interior retainers (360, 362, 364) may be modified, varied, substituted, or supplemented will be apparent to those of ordinary skill in the art in view of the teachings herein.

Based on the teachings herein, those of ordinary skill in the art will appreciate that several elastomeric materials may be suitable for use with guide cube (304). Such elastomeric materials may be used to form the body of guide cube (304), exterior retainer (350), interior retainers (360, 362, 364) and/or other components of guide cube (304). By way of example only, suitable elastomeric materials may include thermosetting plastics that may require vulcanization, thermoplastic elastomers (e.g. Santoprene™ among others), natural rubber, synthetic rubbers (e.g. ethylene propylene diene M-class-EPDM-among others), and other polymers having suitable elastic properties. Other suitable elastomeric materials will be apparent to those of ordinary skill in the art in view of the teachings herein. Similarly, other suitable properties that materials forming retainers (350, 360, 362, 364) may have will be apparent to those of ordinary skill in the art in view of the teachings herein.

Creating a guide cube (304) having elastomeric retainers (350, 360, 362, 364) may be accomplished in a variety of ways. For example, in creating a guide cube such as guide cube (304) that has elastomeric retainers (350, 360, 362, 364), in some versions a multi-shot molding process may be used where the body of guide cube (304) may be molded from a first material, e.g. a non-elastomeric material, and the elastomeric retainers (350, 360, 362, 364) may be molded from a second material, e.g. an elastic material as described herein or otherwise. In some other versions, elastomeric retainers (350, 360, 362, 364) may be molded or extruded separate from the body of guide cube (304) and then coupled with the body of guide cube (304) by mechanical fastening, chemical adhesive, or other suitable bonding or coupling techniques. For instance, guide cube (304) may be molded of substantially hard plastic material, with slots or recesses formed in guide passageways (316, 318, 320) to receive retainers (350, 360, 362, 364). Retainers (350, 360, 362, 364), being separately formed of an elastomeric material, may then be inserted and secured in these slots or recesses. In some other versions, guide cube (304) with retainers (350, 360, 362, 364) may be molded as a single unitary piece having a uniform composition of elastomeric material. Various other suitable ways in which elastomeric retainers (350, 360, 362, 364) may be incorporated into guide cube (304) before, during, or after manufacturing processes will be apparent to those of ordinary skill in the art in view of the teachings herein.

While retainers (350, 360, 362, 364) have been mainly described herein in the context of guide cube (304), it should be understood that retainers (350, 360, 362, 364) may also be incorporated into any other type of guide cube (104, 104a, 104b, 104c) described herein. Similarly, any feature of any one type of guide cube (104, 104a, 104b, 104c, 304) described herein may be incorporated into any other type of guide cube (104, 104a, 104b, 104c, 304) described herein. Thus, the various features and operability of each guide cube (104, 104a, 104b, 104c, 304) described herein should not be viewed as being taught in isolation relative to the other versions of guide cubes (104, 104a, 104b, 104c, 304) described herein. Various ways in which various features and operabilities of a given guide cube (104, 104a, 104b, 104c, 304) described herein may be incorporated into other guide cubes (104, 104a, 104b, 104c, 304) described herein will be apparent to those of ordinary skill in the art in view of the teachings herein.

As noted above, any guide cube or device (104, 104a, 104b, 104c, 304) described herein may be used in a procedure that includes the use of PEM imaging, BSGI imaging, or any other suitable type of imaging. By way of example only, a guide cube or device (104, 104a, 104b, 104c, 304) may be used with a grid plate (96) that is configured for use in an MRI setting, a grid plate for use in a nuclear/molecular imaging setting, or with some other type of cube holder (e.g., “guide holder”) used in nuclear/molecular imaging or other type of imaging. For instance, a suitable alternative cube holder or “guide holder” may include fewer openings (e.g., one to four) that are configured to receive a guide cube or device (104, 104a, 104b, 104c, 304) as compared to the number of recesses (130) in grid plate (96). Furthermore, a guide cube or device (104, 104a, 104b, 104c, 304) may be used with a biopsy device (14) in conjunction with a full targeting set or with just a biopsy device (14) (e.g., in settings where a radioisotope can be communicated through the biopsy device (14)). It should also be understood that a guide cube or device (104, 104a, 104b, 104c, 304) may be used just with a radioisotope, without necessarily involving any biopsy device (14). For instance, a radioisotope may be provided on or through an implement that has a sharp tip, and the implement may be inserted through the guide cube or device (104, 104a, 104b, 104c, 304). Still other various settings and combinations in which a guide cube or device (104, 104a, 104b, 104c, 304) may be used will be apparent to those of ordinary skill in the art in view of the teachings herein.

While several guide cubes have been discussed in detail above, it should be understood that the components, features, configurations, and methods of using the guide cubes discussed are not limited to the contexts provided above. In particular, components, features, configurations, and methods of use described in the context of one of the guide cubes may be incorporated into any of the other guide cubes. One merely exemplary additional feature that may be provided in any of the guide cubes described herein is one or more ridges on one or more external faces of the cube. Such ridges may be substantially rigid, elastomeric, or have any other suitable properties. Such ridges may provide a more secure fit between a cube and grid (e.g., reducing the likelihood that that the guide cube will undesirably fall out of the grid plate), may permit a single cube to be inserted in different grids having differently sized openings, and/or may provide other results. Still other additional and alternative suitable components, features, configurations, and methods of using the guide cubes will be apparent to those of ordinary skill in the art in view of the teachings herein.

Versions of the present invention have application in conventional endoscopic and open surgical instrumentation as well as application in robotic-assisted surgery.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A guide device for guiding a medical instrument relative to a patient, the guide device being usable with a first plate and a second plate, wherein the first plate has a plurality of apertures, wherein the second plate and the first plate are adjustable to secure a portion of the patient, wherein the guide device is configured to be coupled with a selected one of the apertures of the first plate, the guide device comprising:
a. a body defined by at least one surface, wherein the at least one surface comprises a generally proximal portion of the body and a generally distal portion of the body;
b. at least one passageway, wherein the at least one passageway extends from the generally proximal portion through the body and to the generally distal portion, wherein the at least one passageway is configured to receive at least a portion of the medical instrument; and
c. a first retainer operatively configured to deform in response to a force exerted on the first retainer, wherein the first retainer is configured to engage selected one of the medical instrument or the first plate, wherein the first retainer is configured to resist relative movement between the body and the selected one of the medical instrument or the first plate upon engagement of the retainer with the selected one of the medical instrument or the first plate.

2. The guide device of claim 1, wherein the first retainer comprises an external retainer extending outwardly from the body, wherein the first retainer is configured to engage the first plate when the guide device is inserted in a selected one of the apertures of the first plate.

3. The guide device of claim 2, wherein the body forms a cube shape having corners.

4. The guide device of claim 3, wherein the first retainer extends along a plane passing through diagonally opposed corners of the cube shape.

5. The guide device of claim 2, further comprising a second retainer, wherein the second retainer is operatively configured to deform in response to a force exerted on the second retainer.

6. The guide device of claim 5, wherein the second retainer is positioned within the at least one passageway.

7. The guide device of claim 6, wherein the first retainer and the second retainer are both provided by a homogenous continuum of material.

8. The guide device of claim 1, wherein the first retainer comprises an elastomeric wiper.

9. The guide device of claim 7, wherein the first retainer comprises has a first angled face and a second angled face, wherein the first and second angled faces together present a tapered configuration.

10. The guide device of claim 1, wherein the first retainer comprises an internal retainer positioned within the at least one passageway, wherein the first retainer is configured to engage a medical instrument inserted through the at least one passageway.

11. The guide device of claim 10, wherein the at least one passageway defines an internal circumference spanning 360 degrees, wherein the first retainer spans no more than approximately 180 degrees of the internal circumference of the at least one passageway.

12. The guide device of claim 1, wherein the at least one passageway comprises a plurality of passageways, wherein the first retainer extends into at least one passageway of the plurality of passageways.

13. The guide device of claim 12, wherein the plurality of passageways comprise first, second, and third passageways, the guide device further comprising second and third retainers, wherein the first retainer extends into the first passageway, wherein the second retainer extends into the second passageway, wherein the third retainer extends into the third passageway.

14. The guide device of claim 12, wherein the guide device is rotatable about two axes to position a selected one of the passageways to a selected orientation.

15. The guide device of claim 1, wherein the guide device is insertable within the selected one of the plurality of apertures of the first plate, wherein the apparatus further comprises a grounding structure operatively configured to restrict the depth of insertion of the guide device relative to the first plate.

16. The guide device of claim 1, wherein the guide device comprises a plurality of passageways extending through the body, wherein at least two of the passageways are generally parallel to each other.

17. The guide device of claim 16, wherein the plurality of passageways comprise a central passageway, a corner passageway, and an off-center passageway, wherein the central passageway is generally perpendicular to the corner passageway and the off-center passageway.

18. A guide device insertable along a first direction into a grid plate for guiding a medical instrument relative to a patient, the guide device comprising:
a. a body defined by at least four faces, wherein the at least four faces comprise first and second opposing faces and third and fourth opposing faces;
b. a plurality of passageways passing through the body, wherein a first passageway of the plurality of passageways extends from the first face to the second face, wherein a second passageway of the plurality of passageways extends from the third face to the fourth face, wherein each of the passageways is configured to receive at least a portion of the medical instrument inserted along the first direction in accordance with a selected rotational position of the body;
c. a first retainer extending outwardly from the body, wherein the first retainer is configured to engage the grid plate and resist movement of the body relative to the grid plate along a second direction that is opposite to the first direction; and
d. a second retainer extending inwardly into the first passageway, wherein the second retainer is configured to engage the medical instrument and resist movement of the medical instrument along the second direction.

19. The guide device of claim 18, further comprising a third retainer extending inwardly into the second passageway.

20. A method of using a guide device to guide a medical instrument relative to a patient, wherein the guide device comprises a distal portion, a proximal portion, an internal passageway, an exterior wiper retainer, and an interior wiper retainer, the method comprising the steps of:
a. positioning a grid plate adjacent to the patient, wherein the grid plate defines a plurality of apertures;
b. inserting the distal portion of the guide device distally into a selected aperture of the grid plate, wherein the exterior wiper retainer of the guide device folds proximally against the grid plate in response to the act of inserting the distal portion of the guide device, wherein the proximally folded exterior wiper retainer resists proximal movement of the guide device relative to the grid plate; and
c. inserting a portion of the medical instrument distally into the internal passageway, wherein the interior wiper retainer of the guide device folds distally against the inserted portion of the medical instrument in response to the act of inserting the portion of the medical instrument, wherein the distally folded interior wiper retainer resists proximal movement of the portion of the medical instrument relative to the guide device.
